(19) United States
(12) Patent Application Publication
(10) Pub. No.: US 2009/0318692 A1
(43) Pub. Date: Dec. 24, 2009

(54) PROCESS OF MAKING IMATINIB

(76) Inventors: Jakub Castulik, Brno (CZ); Petr Benovsky, Brno (CZ)

Correspondence Address: SYNTHON IP INC, 7130 HERITAGE VILLAGE PLAZA, STE 202, GAINESVILLE, VA, 20155, US

(21) Appl. No.: 12/340,048

(22) Filed: Dec. 19, 2008

Related U.S. Application Data
(60) Provisional application No. 61/008,788, filed on Dec. 22, 2007.

(51) Int. Cl. C07D 403/12; C07D 401/14

(52) U.S. Cl. 544/219; 544/295

(57) ABSTRACT

The present invention provides a process of coupling the compound (2) and compound (3) in the presence of a coupling agent, yielding imatinib of formula (1).

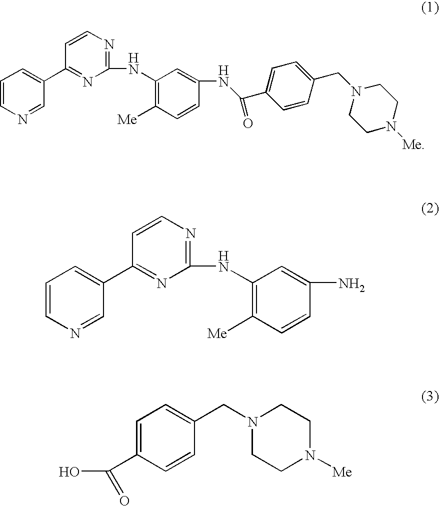

This application claims the benefit of priority under 35 U.S.C. § 119(e) from U.S. provisional patent application Ser. No. 61/008,788, filed Dec. 22, 2007, the entire contents of which being incorporated herein by reference.

### BACKGROUND OF THE INVENTION

The compound imatinib, chemically N-{5-[4-(4-methyl-piperazino-methyl)-benzoylamido]-2-methylphenyl}-4-(3-pyridyl)-2-pyrimidine-amine of the formula (1)

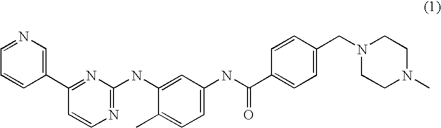

is a pharmaceutically active compound acting as a selective inhibitor of the ABL protein tyrosine kinase. As a (mono)mesylate salt it has been used in a medicament for the treatment of various types of cancer diseases, e.g. under the brand name GLIVEC®. Imatinib has been disclosed in EPB 564409 (U.S. Pat. No. 5,521,184).

A known process for making imatinib comprises, in the last step, a reaction of the N-(2-methyl-5-aminophenyl-4-(3-pyridyl)-2-pyrimidinamine-compound (2)

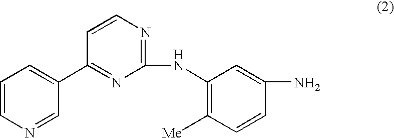

with a reactive derivative of 4-(4-methylpiperazinyl-methyl)benzoic acid-compound (3); advantageously, the reactive derivative is a carbonylhalide compound (3a) [X═Cl]

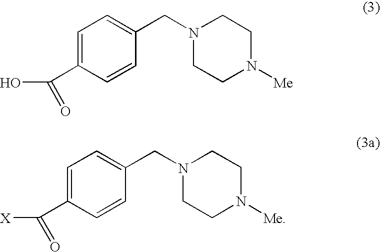

The reaction proceeds in pyridine, under ambient conditions.

An improvement of this process has been disclosed in GB 2398565. In this process, the compound (2) reacts with a dihydrohalide salt of the carbonyl halide (3a) in an inert solvent, e.g. dimethylformamide, to yield a hydrohalide salt of imatinib.

In US 2006/149061, the condensation between (2) and (3a) [X═Cl] proceeds in an inert solvent, e.g. pyridine, with the following steps:

heating the mixture to elevated temperature for a time period sufficient to allow completing the reaction;

cooling the reaction mixture and adjusting the pH;

evaporating the solvent, washing and extracting the residue; and

optionally purifying the reaction product (by a conventional method, preferably by slurrying in ethyl acetate).

The disadvantage of the above processes is that the acid compound (3) cannot be coupled with the compound (2) directly; it must be first converted into a reactive derivative, typically into a carbonylhalide reactive intermediate, which must be isolated, and this intermediate then reacts with (2). Reactive intermediates are, naturally, unstable compounds. Moreover, toxic and hazardous compounds, such as thionyl chloride, are used in the known processes for making these intermediates.

An improvement of the above process is therefore desirable.

### SUMMARY OF THE INVENTION

The present invention provides a process of coupling the compound (2) and compound (3) in the presence of a coupling agent, yielding imatinib of formula (1).

One aspect of the invention provides a process comprising reacting, in a solvent, the compound of formula (2) with the compound of formula (3), or salts thereof, in the presence of a 1,3,5-triazine coupling agent, to form imatinib of formula (1) or a salt thereof. The 1,3,5-triazine coupling agent can be an adduct, especially an adduct formed with a tertiary amine such as N-methylmorpholine. Preferably the 1,3,5-triazine coupling agent is based on 2-chloro-4,6-dimethoxy-1,3,5-triazine of formula (4)

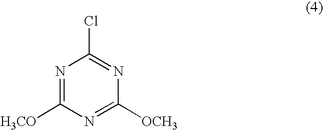

including adducts thereof, etc. In particular, the adduct of formula (6) is a useful coupling agent.

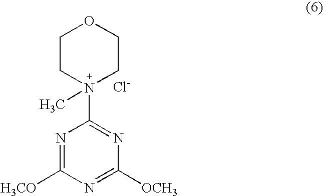

The reaction preferentially proceeds with an acid addition salt of the compound (3), in the presence of a corresponding amount of a base. Advantageously, N-methylmorpholine serves as the base. The formed imatinib is isolated from the reaction mixture and, optionally, is purified and/or converted into a suitable acid addition salt, e.g. imatinib mesylate.

Another aspect of the invention relates to a process for making imatinib, which comprises:

(a) combining a 1,3,5-triazine of formula (4)

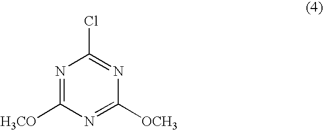

with a tertiary amine, preferably N-methylmorpholine, in a solvent to form a first solvent mixture;
(b) combining a compound of formula (3)

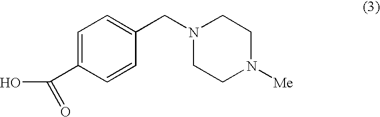

or a salt thereof with said first solvent mixture to form a second solvent mixture;
(c) combining a compound of formula (2)

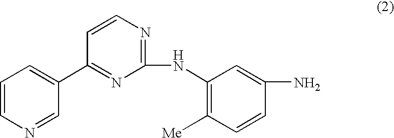

with said second solvent mixture to form a third solvent mixture; and
(d) isolating imatinib or a salt thereof from said third solvent mixture.

An additional aspect of the invention relates to a process for making imatinib from a compound of formula (2) and (3)

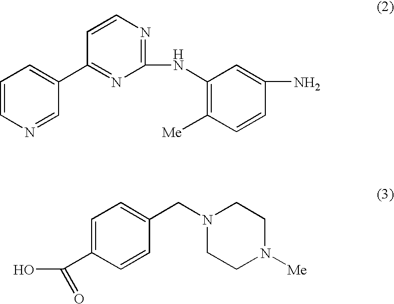

the improvement for which comprises directly coupling said compounds of formula (2) and (3), or salts thereof, with the aid of a 1,3,5-triazine coupling agent; said coupling agent comprising a tertiary amine adduct of 2-chloro-4,6-dimethoxy-1,3,5-triazine of formula (4)

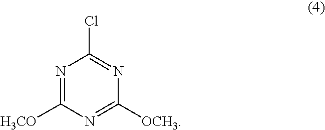

A further aspect of the invention relates to a compound of formula (7)

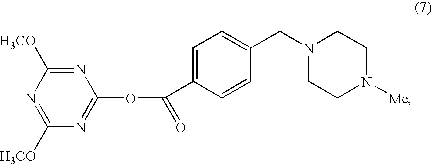

which can be formed by a reaction of the acid (3) with the coupling agent (6). The compound (7) then reacts with the amine compound (2) to form imatinib.

### DETAILED DESCRIPTION OF THE INVENTION

A method for making amides from organic acids and amines using a 1,3,5-triazine compound as a coupling agent is, in general, known in the art. In this respect, cyanuric chloride (2,4,6-trichloro-1,3,5-triazine) is often used as a cheap and efficient agent.

Accordingly, the possibility to use various 1,3,5-triazines as coupling agents in the amidation reaction, wherein the acid component was the compound of the formula (3), and the amine component was the compound of the formula (2), was investigated. The suitable coupling agent should allow mediation of the amide-forming reaction under mild conditions, with high degree of conversion and with minimal amount of side products.

In this respect, it was found that the most suitable 1,3,5-triazine coupling agent is based on 4-chloro-2,6-dimethoxy-1,3,5-triazine (also referred to herein as CDMT) of the formula (4). In an advantageous mode, the agent is used in the form of an adduct with a stoichiometric amount of a suitable tertiary amine; more advantageously, such tertiary amine is N-methylmorpholine, (also referred to herein as NMM) of the formula (5). In this specific case, the adduct has the structure corresponding to the formula (6).

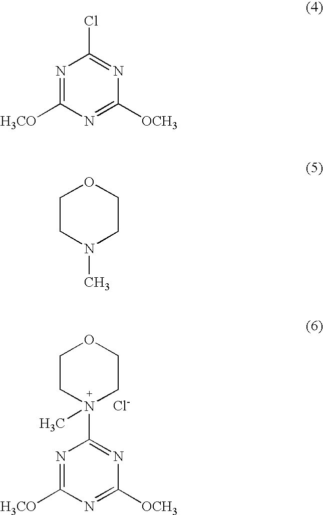

The compound (6) can be obtained as a stable crystalline compound that may be prepared in an extra step or used from a commercial source. In an alternative mode, the adduct (6) may be prepared in situ, before or after the reactants (2) and (3) are charged into the reaction mixture. Advantageously, the adduct is made prior to adding the reactants.

The selected coupling agent fulfills the goals specified above. In essence, the amide-forming reaction proceeds under mild conditions, provides higher yields than the corresponding art-known reaction between the amine (2) and the carbonylhalide (3a), provides higher purity of the crude product (1) and avoids the step of making the carbonylhalide compound using toxic and irritating thionylchloride in the reaction. In particular, formation of side products is minimized, when comparing the composition of the reaction mixture with that as obtained after the reaction of the amine (2) with the carbonylchloride (3a) under the art-known conditions as well as after the reaction of the amine (2) with the compound (3a) that was made in situ.

In an advantageous mode, the starting acid of the formula (3) is used in a form of an acid addition salt; such form is more stable than the acid itself. Suitable salt of the compound (3) is a dihydrochloride hemihydrate, e.g., 4-(4-methylpiperazinyl-methyl)benzoic acid dihydrochloride hemihydrate, which is a stable crystalline compound. Such salt form is commercially available. If the acid is used in the form of a salt, corresponding molar amount of a base should be added into the reaction mixture to neutralize the acid anion. Suitably, the same tertiary amine as used for making the coupling agent (i.e. NMM) may be used as the base, but this is not required.

The amine of formula (2) may be made according to the process known in the art or may be obtained from commercial sources. Similarly as the acid (3), the amine of formula (2) may be employed as an acid addition salt, under the same precautions.

The coupling agent facilitates the coupling of the acid (3) with the amine (2) resulting in an amide. This reaction can have sub-steps. In particular, the acid (3) and the adduct (6) form, in situ, an active ester of formula (7),

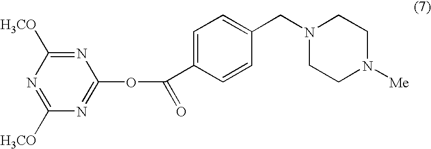

which immediately reacts with the amine (2), when present, in the desired amide-forming reaction. This ester need not be isolated from the reaction mixture. However, it is not excluded that it is isolated; e.g. when prepared in the absence of the amine (2). For purposes of the present invention, the reaction of (7) with amine (2) is considered to be a part of the reaction of (2) with (3) in the presence of the coupling agent. The compound (7) may be formed and/or isolated as an acid addition salt, e.g. as a hydrochloride.

In a suitable order of charging the reagents, first the 1,3,5-triazine compound and the tertiary amine are charged, then the acid (3), and finally the amine (2). The components (2) and (3) may be charged in an isolated state or in a solution.

The reaction proceeds in a solvent, which is advantageously a polar solvent. Suitable solvent is, e.g., acetonitrile, dimethyl formamide, dimethylsulfoxide, tetrahydrofuran, ethyl acetate, water and combinations thereof.

The whole coupling reaction proceeds under stirring at a temperature close to ambient or slightly elevated (20°-50° C.) for a time period sufficient to allow completing the reaction. The course of reaction may be monitored by a suitable analytical technique and the reaction may be terminated in proper time.

The resulting crude imatinib is isolated from the reaction mixture by conventional means, for instance by precipitation, extractions and/or combinations of both. The crude imatinib may be purified by known procedures, e.g. by a recrystallization from a suitable solvent.

Within isolation and/or purification process, imatinib may be converted into, and isolated as, a suitable or desired acid addition salt, e.g. imatinib mesylate.

The invention is further illustrated by the following examples but is not limited thereto.

### EXAMPLE

### Example 1

3 g of 4-chloro-2,6-dimethoxy-1,3,5-triazine were mixed with 100 ml of acetonitrile at 20-25° C. forming solution.

5.2 ml of N-methylmorpholine were added via syringe to the reaction mixture Precipitation of white solid was observed after 1 minute of stirring. Suspension was stirred at 20-25° C. for 1 h.

5 g of 4-(4-methylpiperazinyl-methyl)benzoic acid dihydrochloride hemihydrate were added to the reaction mixture. All solid material was completely dissolved after 10 minutes forming slightly yellow clear solution.

4 g of N-(2-methyl-5-aminophenyl-4-(3-pyridyl)-2-pyrimidinamine were subsequently added to the reaction mixture forming an orange solution. Precipitation of a solid material started after 5 minutes of stirring at 20-25° C. Suspension was stirred at 20-25° C. for 4 hours.

60 ml of 1M aqueous solution of NaOH were added to reaction mixture and layers were separated. Organic layer was then stirred at 20-25° C. and precipitation of solid material was observed after 10 minutes. Suspension was stirred for additional 2 h.

Precipitated solid material was separated by suction and washed with 2×20 ml of acetonitrile.

Yield: 80%

Purity: 98%

### Example 2

6 g of 2-chloro-4,6-dimethoxy-1,3,5-triazine were mixed with 200 ml of acetonitrile at 20-25° C. forming slightly opalescent solution.

10.4 ml of N-methylmorpholine were added and solution turned clear. Precipitation of white solid was observed after 1 minute of stirring. Suspension was then stirred at 20-25° C. for 1 h.

10 g of 4-(4-methylpiperazinyl-methyl)benzoic acid dihydrochloride hemihydrate dissolved in 50 ml of water were subsequently added to the reaction mixture at 20-25° C. Suspension turned to a clear homogeneous solution after 15 minutes of vigorous stirring. Reaction mixture was stirred for 0.5 h and 7.92 g of solid N-(2-methyl-5-aminophenyl-4-(3-pyridyl)-2-pyrimidinamine were added at once forming orange solution. Stirring at 20-25° C. was continued. Reaction was stopped after 3 h and 30 ml of 20% aqueous solution of NaOH were added to the reaction mixture to achieve pH=9-10. It was stirred at 20-25° C. for 1 h and then at 10° C. (bath) for 0.5 h. Solid material was separated by suction and washed with water extensively (c. 100 ml) giving 31.45 g of wet material. It was dried at 20-80° C. (20-60 mbar) for 3 h yielding 12 g of desired product (85% yield) with purity 99%.

Each of the patents, patent applications, and journal articles mentioned above are incorporated herein by reference. The invention having been described it will be obvious that the same may be varied in many ways and all such modifications are contemplated as being within the scope of the invention as defined by the following claims.

We claim:

1. A process which comprises reacting, in a solvent, the compound of formula (2) 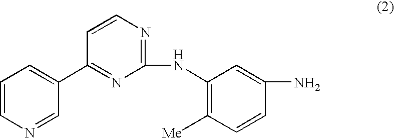
with a compound of formula (3) or a salt thereof 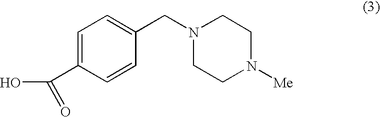
in the presence of a 1,3,5-triazine coupling agent, to form imatinib of formula (1) 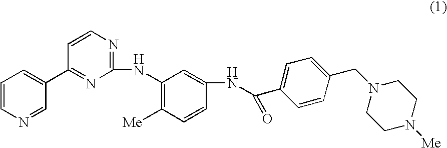
or a salt thereof.

2. The process according to claim 1, wherein said triazaine coupling agent comprises 2-chloro-4,6-dimethoxy-1,3,5-triazine of formula (4) 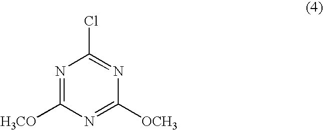
or an adduct thereof with a tertiary amine.

3. The process according to claim 2, wherein said coupling reagent is an adduct and said tertiary amine is N-methylmorpholine of formula (5)

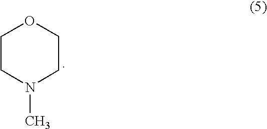

4. The process according to claims 2, wherein said coupling agent is a compound of formula (6)

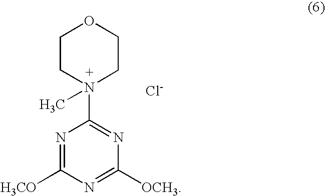

5. The process according to claims 1, wherein said reaction proceeds in the presence of a base.

6. The process according to claim 5, wherein the base is N-methylmorpholine.

7. The process according to claims 1, wherein the solvent is selected from the group consisting of acetonitrile, dimethyl formamide, dimethylsulfoxide, tetrahydrofuran, ethyl acetate, water, and combinations thereof.

8. A process which comprises reacting in a solvent a compound of formula (7) 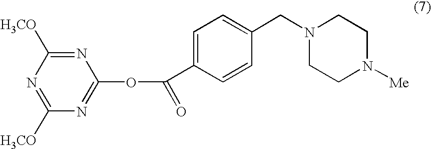
with a compound of formula (2) 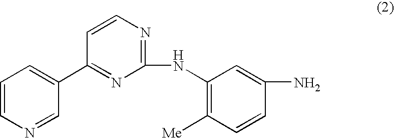
to form imatinib or a salt thereof.

9. The process according to claim 8, wherein said compound of formula (7) was formed in situ in said solvent.

10. The process according to claim 9, which further comprises combining a compound of formula (6) 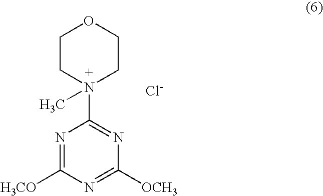
with a compound of formula (3) 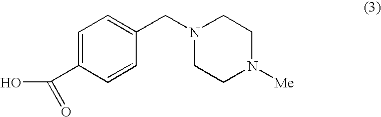
or a salt thereof, in said solvent to form said compound of formula (7).

11. A process which comprises:
(a) combining a 1,3,5-triazine of formula (4) 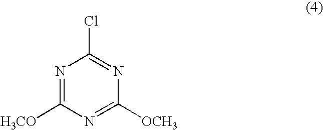
with a tertiary amine in a solvent to form a first solvent mixture;
(b) combining a compound of formula (3) 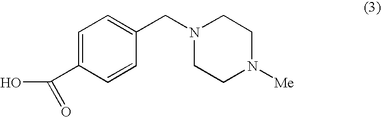
or a salt thereof with said first solvent mixture to form a second solvent mixture;
(c) combining a compound of formula (2) 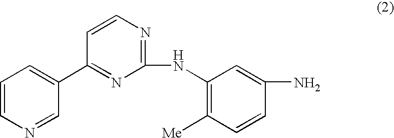
with said second solvent mixture to form a third solvent mixture; and
(d) isolating imatinib or a salt thereof from said third solvent mixture.

12. The process according to claim 11, wherein said tertiary amine in step (a) is N-methylmorpholine

13. In a process for making imatinib from a compound of formula (2) and (3) 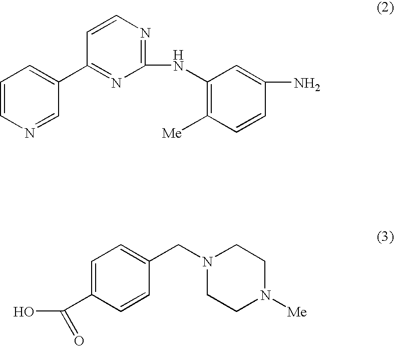
the improvement for which comprises directly coupling said compounds of formula (2) and (3), or salts thereof, with the aid of a 1,3,5-triazine coupling agent; said coupling agent comprising a tertiary amine adduct of 2-chloro-4,6-dimethoxy-1,3,5-triazine of formula (4)

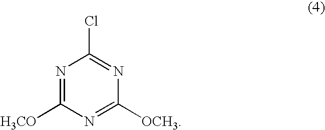

14. The process according to claim 13 wherein said coupling agent is a compound of formula (6)

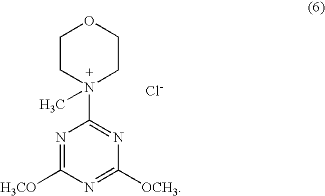

15. A compound of the formula (7) or an acid addition salt thereof [chemical structure]
